SEQUENCE LISTING (1 sequence)

SEQ ID NO: 1 - DNA, 52 bp, Artificial Sequence
  Synthetic Sequence
gggaaatgtactcagtagctatgcaanttagaatgggcagaaagccagaaag

(19) United States
(12) Patent Application Publication
(10) Pub. No.: US 2012/0309639 A1
(43) Pub. Date: Dec. 6, 2012

(54) Compositions and Methods for Diagnosing Genome Related Diseases and Disorders

(76) Inventors: Hakon Hakonarson, Malvern, PA (US); Kai Wang, Los Angeles, CA (US); Zhi Wei, Princeton, NJ (US)

(21) Appl. No.: 13/499,515

(22) PCT Filed: Oct. 8, 2010
(86) PCT No.: PCT/US10/51972

§ 371 Date: Aug. 14, 2012

Related U.S. Application Data
(60) Provisional application No. 61/249,711, filed on Oct. 8, 2009.

(51) Int. Cl. G06F 19/24; C40B 40/06; A61B 5/00; C40B 30/04

(52) U.S. Cl. 506/9; 506/16; 702/19

(57) ABSTRACT

Compositions, methods, and kits for diagnosing genetic disorders such as type I diabetes are disclosed.

### RELATED DISEASES AND DISORDERS

This application claims priority under 35 U.S.C. §119(e) to U.S. Provisional Patent Application No. 61/249,711, filed on Oct. 8, 2009. The foregoing application is incorporated by reference herein.

### FIELD OF THE INVENTION

The present invention relates to the field of diagnosing genetic diseases and disorders. More specifically, the invention provides compositions and methods for diagnosing type I diabetes.

### BACKGROUND OF THE INVENTION

Several publications and patent documents are cited throughout the specification in order to describe the state of the art to which this invention pertains. Each of these citations is incorporated herein by reference as though set forth in full.

Genome-wide association studies (GWAS) have been successfully employed to interrogate the genetic architecture of common and complex diseases (McCarthy et al. (2008) Nat. Rev. Genet., 9:356-369). Unlike traditional linkage and candidate gene association studies, GWAS have enabled human geneticists to examine a wide range of complex phenotypes, and have allowed the confirmation and replication of previously unsuspected susceptibility loci. Some of the more notable examples of success include dozens of susceptibility loci now known to modify individual disease risk of type 2 diabetes (T2D) (Zeggini et al. (2008) Nat. Genet., 40:638-645), type 1 diabetes (T1D) (Cooper et al. (2008) Nat. Genet., 40:1399-1401; Barrett et al. (2009) Nat Genet., 41:703-7), Crohn's disease (CD) (Barrett et al. (2008) Nat. Genet., 40:955-962), as well as loci influencing polygenic traits such as height (Weedon et al. (2008) Nat. Genet., 40:575-583; Gudbjartsson et al. (2008) Nat. Genet., 40:609-615; Lettre et al. (2008) Nat. Genet., 40:584-591), body mass index (Willer et al. (2009) Nat. Genet., 41:25-34) and dyslipidemia (Kathiresan et al. (2009) Nat. Genet., 41:56-65). However, for many conditions, these variants still explain only a small proportion of individual differences in disease predisposition or phenotypic diversity; for example, the 54 validated loci that influence human height collectively only explain 4-6% of variation in the trait after adjustment of age and sex (Aulchenko et al. (2009) Eur. J. Hum. Genet., 17:1070-5), and the 31 validated susceptibility loci for Crohn's disease collectively only explains 20% of the genetic risk variance (Barrett et al. (2008) Nat. Genet., 40:955-962). Identifying most of the remaining genetic variance still represents a challenge, albeit tractable, for the foreseeable future.

Besides identifying genes influencing disease susceptibility or phenotypic variation, another often suggested utility of GWAS is that these discoveries will facilitate implementation of personalized medicine, in which preventive and therapeutic interventions for complex diseases are tailored to individuals based on their genetic make-up, as can be determined by genome-wide genotyping profiles on a SNP-based array. The latter promise is now routinely used for certain monogenic disorders where the underlying genetic factor has already been characterized. However, for common and complex diseases, where multiple loci work together to increase disease risk, the application of personalized medicine may not be as straightforward. In fact, several studies have been recently published on the assessment of risk for common diseases using multiple genetic variants (reviewed in Janssens et al. (2008) Hum. Mol. Genet., 17:R166-173; Wray et al. (2007) Genome Res., 17:1520-1528; Wray et al. (2008) Curr. Opin. Genet. Dev., 18:257-263; Jakobsdottir et al. (2009) PLoS Genet 5: e1000337; Kraft et al. (2009) N. Engl. J. Med., 360:1701-1703). However, a consistent theme from these studies is that disease assessment methods so far show limited predictive value, and the performance of these studies is far below what would be considered clinically feasible or practical. For example, at least four studies have been conducted to use several “validated” variants for risk prediction of type 2 diabetes (T2D) (Weedon et al. (2006) PLoS Med., 3:e374; Vaxillaire et al. (2008) Diabetes 57:244-254; Lango et al. (2008) Diabetes 57:3129-3135; van Hoek et al. (2008) Diabetes 57: 122-3128). The AUC (area under the receiver operating characteristic curve) scores for the T2D studies range from 0.55 to 0.60, indicating that the prediction is only slightly better than chance. However, they also indicate that the use of more markers should lead to improvement of predictive performance. A more recent study on multiple human diseases reported slightly higher AUC scores for Age-related Macular Degeneration (AMD) and Crohn's Disease (CD), but the results are still largely negative; in fact, the authors cautioned that the scientific community should avoid “overstating the value of association findings in terms of personalized medicine” (Jakobsdottir et al. (2009) PLoS Genet 5: e1000337). Other similar negative studies have been conducted for a variety of human diseases and complex traits, such as height (Aulchenko et al. (2009) Eur. J. Hum. Genet., 17:1070-5), coronary heart disease (van der Net et al. (2009) Am. Heart J., 158:105-110) and cardiovascular diseases (Paynter et al. (2009) Ann. Intern. Med., 150:65-72), although positive studies have been reported for AMD when combining genetic, demographic, and environmental variables (Seddon et al. (2009) Invest. Ophthalmol. Vis. Sci., 50:2044-2053). Altogether, these observations have triggered concern about the potential value of individual-based disease risk assessment, at least for the time being.

### SUMMARY OF THE INVENTION

In accordance with the instant invention, methods of determining a set of markers predictive for a disease or disorder are provided. The instant invention also provides methods of diagnosing a disease or disorder in a patient using the set of predictive markers.

In accordance with another aspect of the instant invention, microarrays comprising the set of predictive markers are provided. Kits comprising the microarrays are also provided.

### BRIEF DESCRIPTION OF THE DRAWINGS

FIG. 1 provides graphs of the performance of risk assessment models trained on the WTCCC-T1D dataset. For both the CHOP/Montreal-T1D and the GoKind-T1D datasets, the SVM (support vector machine) algorithm consistently outperforms LR (logistic regression), and the best performance is achieved when SNPs were selected using P-value cutoff of 1×10⁻⁶ or 1×10⁻⁵.

FIG. 2 provides graphs of the performance of risk assessment models trained on the CHOP/Montreal-T1D dataset. For both the WTCCC-T1D and the GoKind-T1D datasets, the SVM (support vector machine) algorithm consistently outperforms LR (logistic regression), and the best performance is achieved when SNPs were selected using P-value cutoff of 1×10⁻⁶ or 1×10⁻⁵.

FIG. 3 provides a graph of the specificity of the SVM-based risk assessment models. The risk assessment models were parameterized on the WTCCC-T1D dataset and evaluated on other disease cohorts from WTCCC, including bipolar disorder (BD), coronary heart disease (CAD), Crohn's disease (CD), hypertension (HT), rheumatoid arthritis (RA), and type 2 diabetes (T2D). The specificity measure was calculated with default cutoff of zero point. Except for RA, the specificity measures of the prediction model are comparable for other diseases as that for the control subjects.

FIG. 4 provides an illustration on how positive predictive value (PPV) and negative predictive value (NPV) vary with respect to disease prevalence in a testing population. The figure is based on sensitivity and specificity estimates from WTCCC-T1D data set on CHOP-T1D data when P<1×10⁻⁵ is used. The three vertical lines represent three different scenarios of clinical testing, with disease prevalence of 0.4%, 6% and 13%, respectively.

FIGS. 5A-5J provide a list of 478 SNPs used in the Example. Sequences of the SNPs can be found at www.ncbi.nlm.nih.gov/pubmed/ in the SNP database. For example, rs2269241 yields the sequence GGGAAATGTACTCAGTAGCTATGCAA [A/G] TTAGAATGGGCAGAAAGCCAGAAAG (SEQ ID NO: 1), where G is the ancestral allele.

### DETAILED DESCRIPTION OF THE INVENTION

Since the majority of risk factors for human diseases or complex traits have yet to be identified, the failure of previous studies on predicting individual disease risk is not unexpected. First, all these studies have investigated only a limited number of susceptibility variants that were confirmed in previous GWAS. As previously discussed, these validated susceptibility loci typically only explain a small proportion of the genetic risk underlying phenotypic variance. Therefore, the omission of the vast majority of genuine susceptibility loci that are yet to be validated from GWAS precludes success, since most of the informative markers are absent from prediction model. For example, none of the four T2D risk assessment studies (Weedon et al. (2006) PLoS Med., 3:e374; Vaxillaire et al. (2008) Diabetes 57:244-254; Lango et al. (2008) Diabetes 57:3129-3135; van Hoek et al. (2008) Diabetes 57: 122-3128) used more than 20 SNPs. It cannot be expected that any such study would yield satisfactory results from a handful of loci, which collectively explain only a minor fraction of disease risk. It is acknowledged that the reason why most genetic variants have not been identified is because the first generation of GWAS were powered to detect variants of large effect sizes. With the ever increasing sample sizes in GWAS, more loci will be discovered and validated in the future. Second, only relatively simple statistical approaches, such as additive genotype scores (unweighted or weighted number of risk alleles) or logistic regression assuming independence between variants, have been applied in previous studies. These approaches, although widely used in statistical genetics, do not take into account the complex relationships or interactions between multiple loci contributing to disease risk. In fact, regression analysis is optimized for the purpose of estimating the effects of predictor variables, unlike other more sophisticated machine-learning approaches (especially maximum-margin approaches) for the sole purpose of classification or discrimination. Finally, the whole-genome genotype data in the diseases from these previous studies are probably “overly” complex, with the genetics per se possibly only explaining a small proportion of disease incidence, unless coupled with other factors such as environmental exposures. For example, T2D has a heritability estimate of ˜50% (Stumvoll et al. (2005) Lancet 365: 1333-1346) while T1D has a much stronger familial component, with a heritability estimate of ˜90% (Hyttinen et al. (2003) Diabetes 52:1052-1055). Therefore, it can be expected that whole-genome genotype-based disease risk assessment would operate better in T1D than in previous studies of T2D. Altogether, it is clear that alternative routes should be taken for a better evaluation of individual disease risk assessment.

Herein, the issues discussed above have been addressed. First, rather than cherry-picking a few known susceptibility loci for disease risk assessment, an entire list of markers reaching a pre-defined statistical threshold for association with a disease (for example, P<1×10⁻⁵) were utilized, even if the majority of SNPs in that list have not been confirmed to be genuine susceptibility loci. There is no doubt that some false positive hits will arise, but it is shown that the computational approaches used are in fact robust with the inclusion of these non-contributing markers when also taking advantage of other markers that are already established to be associated with the disease. Second, a Support Vector Machine (SVM) (Vapnik, V. (1999) The Nature of Statistical Learning Theory, 2n^{d} Edition, Springer), a well-developed machine-learning technique in computer science, has been utilized. Unlike traditional “number of risk alleles” approach or logistic regression assuming independence between markers, this approach can both optimize prediction modeling and take advantage of potential interactions between markers to achieve the optimal binary predictive power. Finally, T1D was used as an example for disease assessment. Unlike other common diseases, such as T2D or coronary heart disease, a large fraction of variance of genetic risk is already known for T1D. Indeed, over 50% of the genetic susceptibility to T1D pathogenesis can be explained by risk alleles in the major histocompatibility complex (MHC) region alone, while the remaining genetic contribution has been attributed to variants conferring more moderate risks (Ounissi-Benkalha et al. (2008) Trends Mol. Med., 14:268-275). The availability of multiple GWAS datasets for T1D, including samples from different geographical sites, genotyped at different locations and on different genotyping platforms (Affymetrix Mapping 500K and Illumina® HumanHap550 SNP arrays), allows the unbiased and systematic comparative evaluation of different methods.

Genome-wide association studies (GWAS) have been fruitful in identifying disease susceptibility loci for common and complex diseases. A remaining question is whether individual disease risk can be quantified based on genotype data, in order to facilitate personalized prevention and treatment for complex diseases. Previous studies have typically failed to achieve satisfactory performance, primarily due to the use of only a limited number of confirmed susceptibility loci. Here, it is shown that sophisticated machine-learning approaches with a large ensemble of markers improve the performance of disease risk assessment. A Support Vector Machine (SVM) algorithm was applied on a GWAS dataset generated on the Affymetrix genotyping platform for type 1 diabetes (T1D) and optimized a risk assessment model with hundreds of markers. This model was subsequently tested on an independent Illumina®-genotyped dataset with imputed genotypes (1,008 cases and 1,000 controls), as well as a separate Affymetrix-genotyped dataset (1,529 cases and 1,458 controls), resulting in area under ROC curve (AUC) of ˜0.84 in both datasets. In contrast, poor performance was achieved when limited to dozens of known susceptibility loci in the SVM model or logistic regression model. The instant study indicates that improved disease risk assessment can be achieved by using algorithms that take into account interactions between a large ensemble of markers. Genotype-based disease risk assessment is feasible for diseases, particularly where a notable proportion of the risk has already been captured by SNP arrays.

In this study (see Example), the plausibility of building a classifier and using a large number of SNPs for disease risk assessment was tested on three large T1D datasets. In general, the SVM algorithm achieved satisfactory performance when hundreds of SNPs were included in prediction models, with AUC scores of ˜0.84 for predicting disease risk for T1D in several GWAS datasets. In contrast, the SVM or the LR algorithm achieved only an AUC score of 0.66-0.68 when 45 known T1D susceptibility loci were used. This difference clearly indicates that the predictive value lies in utilizing a large number of SNPs in a sophisticated machine-learning algorithm. On the other hand, a decrease in the predictive accuracy was observed when too many SNPs were used, suggesting an upper bound of the number of SNPs for T1D risk assessment before noises from falsely associated markers lead to degraded performance. However, this upper bound depends on the sample size and the power of the study to rank truly associated SNPs higher than background noises.

One of the major differences between the two classifiers used in the study is in their capability of handling main and interaction effects. SVM takes into account both effects, while LR aims to model linear main effects but ignores interaction. As an example, for a simple interaction model with two risk loci A and B, the disease risk will increase significantly only if A=0 and B=1 or A=1 and B=0. Such interaction can be captured by SVM using various kernels, but not by the simple LR model. In this study, it was observed that SVM outperformed LR by taking into account both main and interaction effects, implying that both genes and their interactions contribute to T1D. This is particularly important for T1D, as many previous studies have already shown that risk from MHC and non-MHC regions accumulates at a rate less than expected from the model of multiplicative effects, and that the relative risks for non-MHC loci are reduced when MHC-related risk is high (Barrett et al. (2009) Nat Genet., 41:703-7). Additionally, it was also found that better modeling of LD structure within MHC (haplotype interactions) play a major role in SVM-based risk modeling. However, these issues have been largely ignored in previous simulation studies on disease risk assessment (Wray et al. (2007) Genome Res., 17:1520-1528; Jakobsdottir et al. (2009) PLoS Genet., 5:e1000337; Daetwyler et al. (2008) PLoS ONE 3: e3395), probably because the appropriate modeling of interaction effect is by itself not well understood. Nevertheless, some previous real-data studies already documented the importance of interactions effects in prediction model for quantitative or qualitative phenotypes: for example, Lee et al. have applied a MCMC approach that takes into account of within and between loci interactions, for phenotype prediction in heterogeneous stock mouse population in a cross-validation design (Lee et al. (2008) PLoS Genet., 4:e1000231). Therefore, while simulation studies are useful in drawing general qualitative conclusions such as that predictive accuracy increases with heritability, their quantitative findings may not be accurate because of the “non-interaction” assumptions they have to make. Although there may be limited understanding of the interaction patterns of variants underlying common and complex diseases, the previous simulation results may not necessarily reflect real-world scenario.

Although the implementation of individual risk assessment in clinical settings will have major economic benefit to the public health at the population level (Khoury et al. (2006) Genet. Med., 8:191-195), the clinical utility of individual risk assessment depends on a few factors that must be taken into account. First, the appropriateness of individual risk assessment is dependent on the genetic etiology of the disease (Kraft et al. (2009) N. Engl. J. Med., 360:1701-1703). Given that the vast majority of the phenotypic variation in T1D can be explained by genetic factors, T1D assessment would have a strong basis for being applied in clinical settings. In fact, HLA-typing, albeit being both costly and imperfect, is now being used in clinical settings for assessing T1D risk for siblings of affected patients. Unlike T1D, where genetic factors are estimated to explain ˜90% of the phenotypic variance, the heritability estimates for T2D are less than 50% (Stumvoll et al. (2005) Lancet 365:1333-1346). Therefore, even if all genetic risk factors are identified ultimately for T2D and a perfect SNP-based prediction model is available for the disease, they would have less impact in a clinical setting than T1D prediction models.

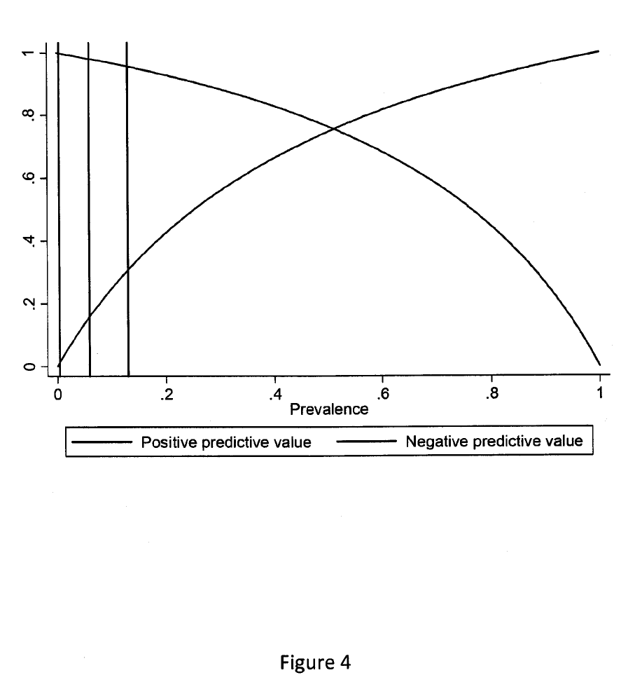

Second, the clinical utility of a risk assessment model depends on the disease prevalence at the particular clinical setting. Using the sensitivity and specificity measures for the WTCCC T1D model on CHOP/Montreal-T1D datasets (FIG. 4), three scenarios of diagnostic testing were evaluated using SNP arrays: (1) general population (disease prevalence=0.4%), (2) siblings of affected patients (disease prevalence=6%), (3) siblings of early-onset patients who developed diabetes before 5 yrs of age (disease prevalence=13%). When a general population is screened by the prediction model, the positive predictive values are relatively modest, indicating that the risk assessment model is not of much utility for population-level screening. However, in a realistic clinical setting, where siblings of affected patients are evaluated, the WTCCC-T1D prediction model achieves a positive predictive value of 16% and a negative predictive value of almost 100%; that is, ˜16% of predicted positive patients will eventually develop the disease, while very few predicted negative patients will develop the disease, with overall accuracy of 93%. Finally, for siblings of early-onset patients, the positive predictive value reaches 31%, while a strong negative predictive value of 96% can still be retained with an overall prediction accuracy of 87%. Although T1D has a large genetic contribution from risk alleles in the MHC region, it is well known that costly HLA-typing per se is not sufficient for T1D risk assessment with high accuracy. Based on these results, low-cost SNP genotyping platforms can replace HLA-typing in assessing T1D risk in clinically relevant settings.

Third, for a given disease, the best assessment model and the most optimal number of predictors (SNPs) may depend on the distribution of effect sizes. Some autoimmune diseases, such as T1D, have major-effect loci (MHC) that explain a large proportion of the genetic risk (˜50% for T1D), with additional moderate-effect loci that explain the remaining proportion. Therefore, for these diseases, the collection of SNPs with P<1×10⁻⁵ in a given GWAS probably already captured the vast majority of the variance for genetic susceptibility, and can lead to good prediction performance. Therefore, diseases such as T1D might represent an extreme example where genotype-based risk assessment is clinically feasible. In contrast, MHC loci play a much less important role or no role in CD or T2D susceptibility, so a much more liberal P-value threshold may be required for SNP selection, to ensure the capture of a large fraction of the genetic risk in prediction models. This step will likely include more markers that are falsely associated with the disease in prediction models, and may dilute the contribution from genuinely associated loci. Taking interception from independent datasets (for example, SNPs with P<0.05 in two GWAS) may be explored for risk assessment on these diseases. Furthermore, diseases such as psychiatric disorders do not appear to even have any major-effect loci that are common, so accurate assessment of disease risk may require even more markers or whole-genome markers.

Finally, besides genetic factors, other risk factors that are specific for each disease need to be accounted for in order to make an accurate risk assessment. Early onset diseases such as T1D may be less dependent on non-genetic factors. In contrast, T2D is a late-onset disease with a range of known environmental risk factors contributing to its pathogenesis, and may be predicted more accurately if such factors are also used. Therefore, a comprehensive disease risk assessment model should try to take into account environmental risk factors, such as diet and smoking habits, as well as other predictor variables such as gender and BMI in order to improve performance. These factors are most likely disease-specific and can be identified from cumulative epidemiological studies on each disease. Notably, the SVM model used in this study can readily take into account additional predictor variables.

In conclusion, the results from recent GWAS have yielded enormous amounts of data that can be mined and utilized for better understanding of human disease, including disease risk assessment using genetic profiles. Although only a small fraction of risk factors for complex diseases have been identified to date, other variants with moderate effects are present in the GWAS data, and a risk assessment algorithm should be able to take advantage of these variants for improved performance. Methods that utilize whole-genome data, rather than a few “validated” susceptibility loci, improve predictive accuracy and have greater impact on health care in the future; for example, by applying personalized intervention strategies on newborns who are at risk of developing T1D. Their risk of developing the disease may be reduced or physicians will be better prepared to treat the disease. This would be feasible if these individuals can be identified from genetic risk profiles, using algorithms (such as the SVM algorithm proposed in this study) with high positive predictive values.

In accordance with the instant invention, methods of determining a set of markers predictive for a disease or disorder are provided. In a preferred embodiment, the disease or disorder has a basis within the genome (i.e., it is not completely determined by environmental factors). For example, it is preferred if genetic factors account for at least 50%, at least 75%, at least 80%, at least 85%, at least 90%, at least 95%, or even 100% of the phenotypic variance. The disease or disorder can be, without limitation, type 1 diabetes, schizophrenia, autism, inflammatory bowel disease such as Crohn's Disease and colitis, inflammatory/autoimmune diseases including but not limited to juvenile rheumatoid arthritis, lupus, celiac disease, and asthma. In a particular embodiment, the disease is type I diabetes. In a particular embodiment, the methods for determining a set of predictive markers comprise 1) obtaining a genome wide association studies dataset; 2) selecting those markers within the dataset that have a P-value of less than 1×10⁻⁶, less than 1×10⁻⁵, or less than 1×10⁻⁴; and 3) applying a support vector machine algorithm to the selected dataset. In one embodiment, the P-value is less than 1×10⁻⁵. The marker may be a SNP, deletion, insertion, rearrangement, recombination, or other alteration to the wild-type sequence.

The instant invention also provides methods of diagnosing a disease or disorder in a patient. In a particular embodiment, the method comprises, 1) obtaining a biological sample from a patient; 2) determining the presence or absence of the predictive markers for the disease or disorder; and 3) applying a support vector machine algorithm to the results obtained in step 2) to predict the disease risk in the patient. In a particular embodiment, step 2) is performed by hybridizing the nucleic acids of the biological sample (optionally amplified (e.g., via PCR)) with the set of predictive markers (e.g., by using a microarray). Upon diagnosing the patient as being at risk or having a particular disease or disorder, the patient may subsequently be treated for the disease or disorder. For example, with regard to type 1 diabetes, the onset of the disease may be delayed or prevented by the administration of insulin (e.g., orally or by inhalation) (see, e.g., Clinical Trials NCT00223613 and NCT00419562 at www.clinicaltrials.gov) and/or the administration of at least one immunosuppressant (e.g., anti-CD20 (rituximab), Mycophenolate mofetil (MMF/CellCept®), and Daclizumab (DZB/Zenapax®).) (see, e.g., Clinical Trials NCT00279305 and NCT00100178).

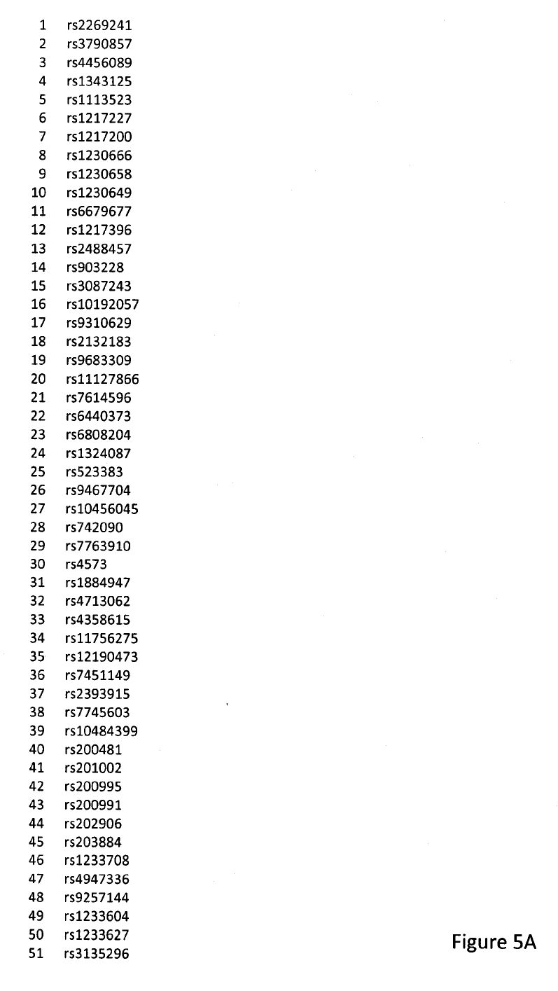

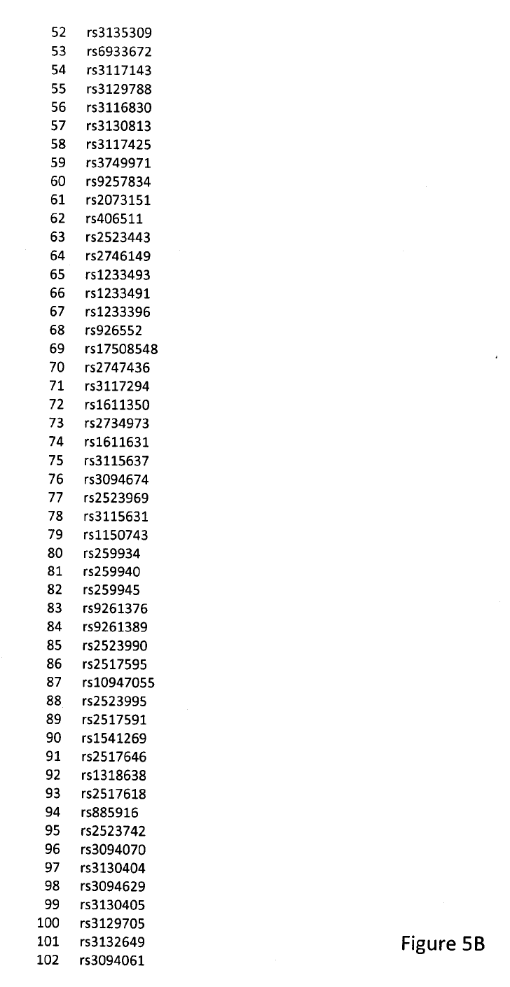

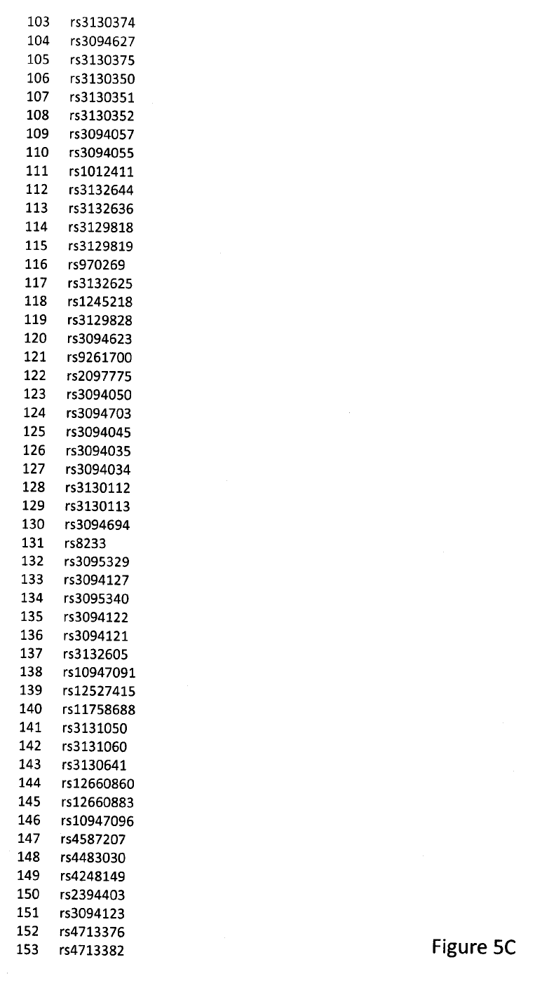

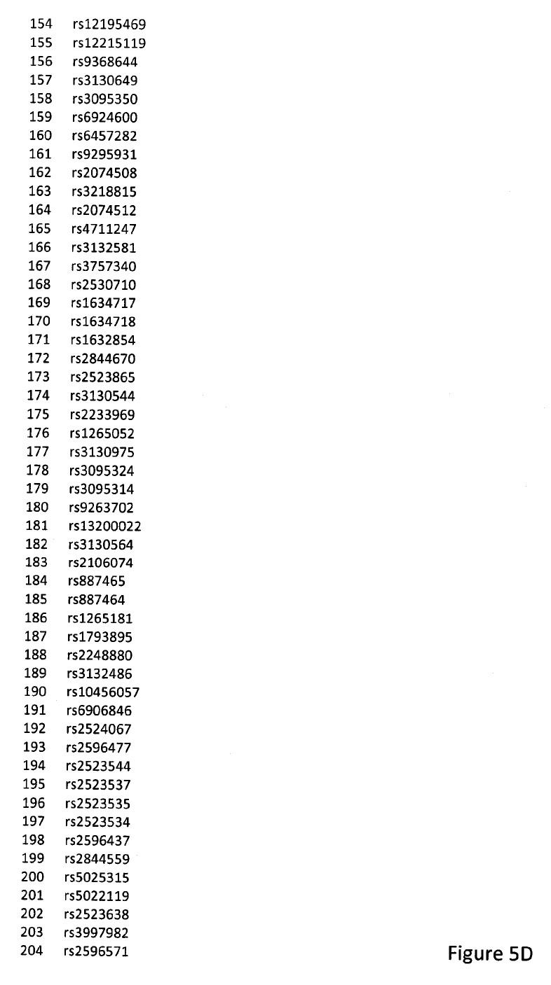

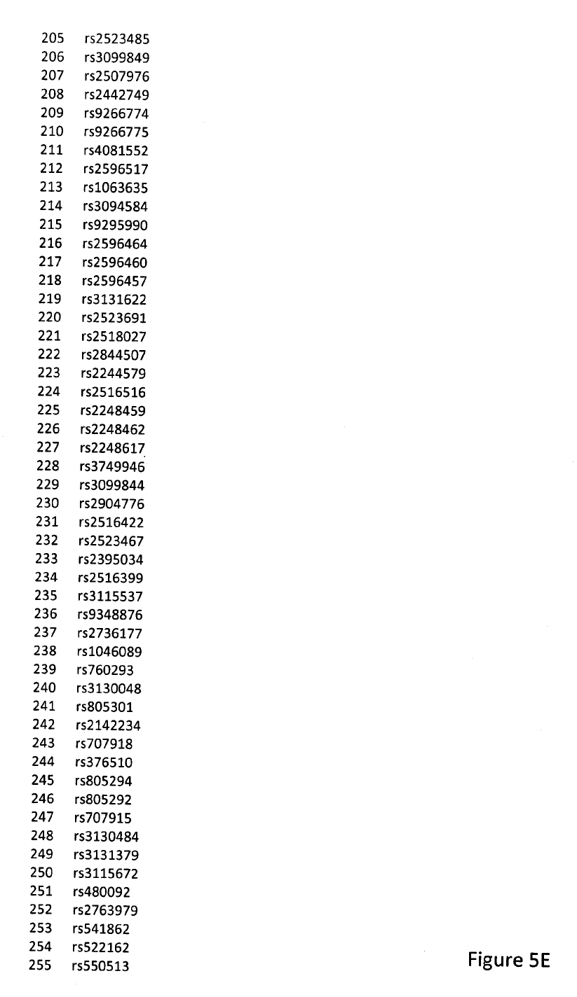

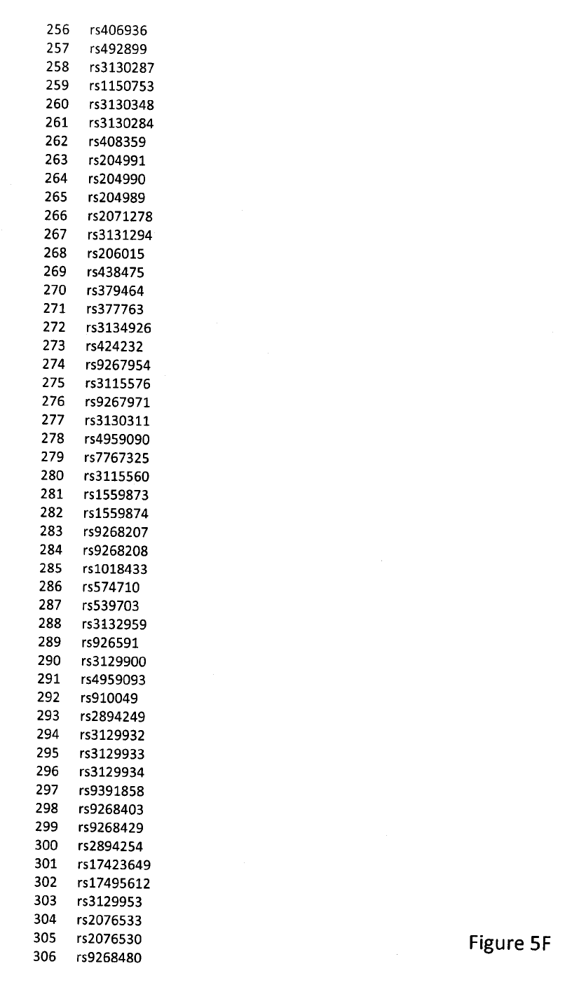

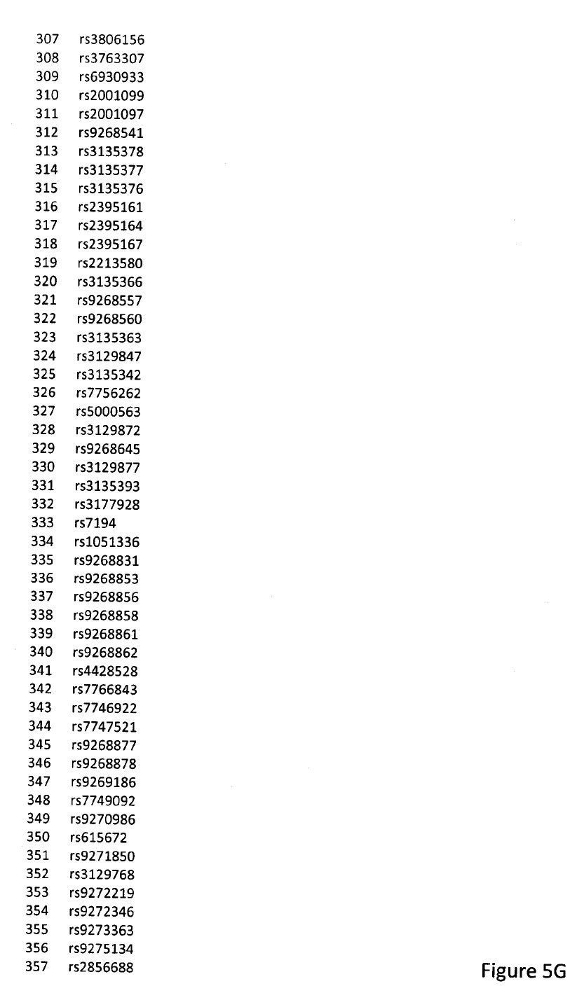

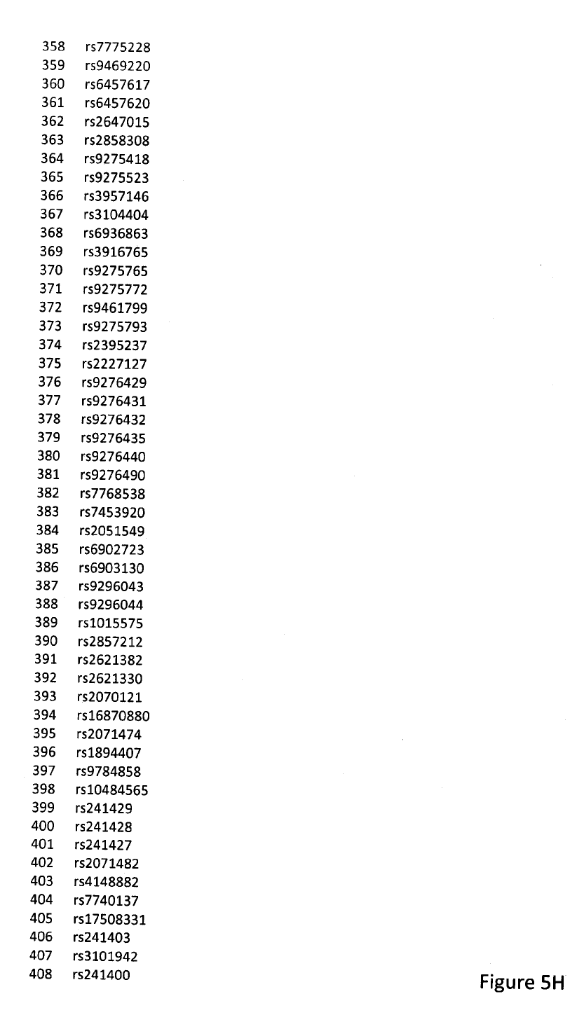

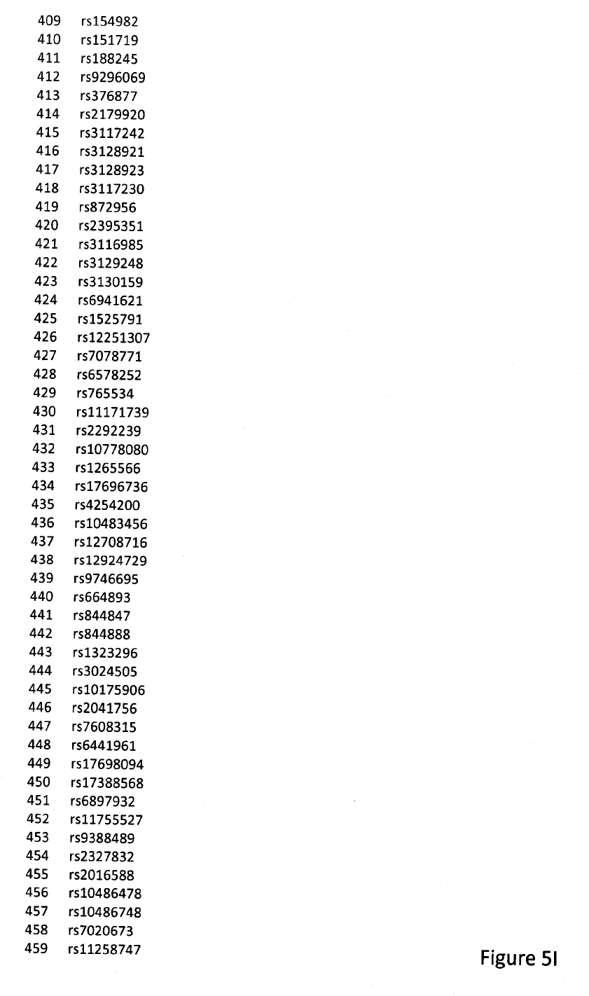

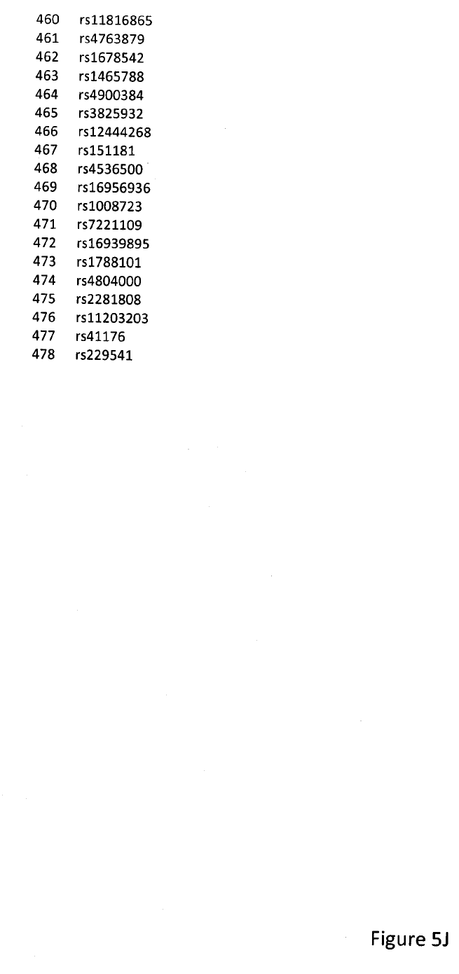

In accordance with another aspect of the present invention, microarrays for diagnosing a disease or disorder (e.g., type I diabetes) are provided. In a particular embodiment, the microarray comprises oligonucleotides probes predictive for the disease (see above) attached to a solid support (e.g., a chip). In a particular embodiment, the microarray comprises oligonucleotide probes which comprise or specifically hybridize to the SNPs presented in FIG. 5. The microarrays may comprise oligonucleotide probes which comprise or specifically hybridize with at least 80%, at least 90%, at least 95%, at least 97%, at least 99%, or all of the 478 SNPs provided in FIG. 5. In a preferred embodiment, the oligonucleotide probes hybridize with the SNPs presented in FIG. 5 to the exclusion of the wild-type sequence (e.g., when considering the hybridization and washing conditions used with the microarray). In a particular embodiment, the oligonucleotide probes are completely complementary to the SNPs provided in FIG. 5. In another embodiment, the oligonucleotide probes comprise or consist of the SNPs provided in FIG. 5 (e.g., the probe may be 20 nucleotides in length and comprise the single nucleotide change of one of the sequence provided in FIG. 5). In yet another embodiment, the oligonucleotide probes are about 10, 15, 20, 25, or 30 to about 40, 50, 75, or 100 nucleotides in length. In a particular embodiment, the oligonucleotide probe is 52 nucleotides in length. In a particular embodiment, the single nucleotide change is towards the middle of the oligonucleotide probe (e.g., within the middle third of the oligonucleotide probe). In another embodiment, the microarray further comprises probes unrelated (e.g., control oligonucleotides) to the disease or disorder (e.g., type 1 diabetes).

In accordance with another aspect of the instant invention, kits for diagnosing type 1 diabetes are provided. The kits may comprise at least one microarray of the instant invention. The kit may further comprise instruction material and/or means for obtaining the biological sample and/or at least one positive control (nucleic acid molecules positive for type 1 diabetes) and/or at least one negative control (nucleic acid molecules negative for type 1 diabetes). In a particular embodiment, the kit comprises instruction material or program for analyzing the microarray and diagnosing whether the subject is at risk for type 1 diabetes. The instructional material or program may be contained on any digital data storage (e.g., a CD) or may be accessible via the interne via a website provided with the kit (optionally password protected).

### Definitions

As used herein, a “biological sample” refers to a sample of biological material obtained from a subject, preferably a human subject, including, without limitation, a tissue, a tissue sample, a cell(s), and a biological fluid (e.g., blood, amniotic fluid, or urine). A biological sample comprising nucleic acids of the subject may be obtained by any method (e.g., buccal swab or biopsy).

As used herein, “diagnose” refers to detecting and identifying a disease in a subject. The term may also encompass assessing, evaluating, and/or prognosing the disease status (progression, regression, stabilization, response to treatment, etc.) in a patient known to have the disease.

As used herein, the term “prognosis” refers to providing information regarding the impact of the presence of a disease on a subject's future health (e.g., expected morbidity or mortality, the likelihood of developing disease, and the severity of the disease). In other words, the term “prognosis” refers to providing a prediction of the probable course and outcome of the disease or the likelihood of recovery from the disease.

As used herein, the term “microarray” refers to an ordered arrangement of hybridizable array elements. The array elements are arranged so that there are preferably at least one or more different array elements, more preferably at least 100 array elements, and most preferably at least 1,000 array elements on a solid support. Preferably, the hybridization signal from each of the array elements is individually distinguishable, the solid support is a chip, and the array elements comprise oligonucleotide probes.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains about 10-100, about 10-50, about 15-30, about 15-25, about 20-50, or more nucleotides, although it may contain fewer nucleotides. The probes herein may be selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target, although they may. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

For instance, one common formula for calculating the stringency conditions required to achieve hybridization between nucleic acid molecules of a specified sequence homology is set forth below (Sambrook et al., 1989):

Tₘ =81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “isolated” may refer to a compound or complex that has been sufficiently separated from other compounds with which it would naturally be associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

The phrase “solid support” refers to any solid surface including, without limitation, any chip (for example, silica-based, glass, or gold chip), glass slide, membrane, bead, solid particle (for example, agarose, sepharose, polystyrene or magnetic bead), column (or column material), test tube, or microliter dish.

The following example is provided to illustrate various embodiments of the present invention. It is not intended to limit the invention in any way.

### EXAMPLE

### Methods

Type 1 Diabetes (T1D) GWAS Dataset from WTCCC

The 500K Affymetrix chip genotype data from WTCCC on ˜1,500 samples from the 1958 British Birth Cohort, ˜1,500 samples from the UK Blood Service Control Group, as well as ˜2,000 samples each from the following disease collections: type 1 diabetes (T1D), type 2 diabetes (T2D), rheumatoid arthritis (RA), inflammatory bowel disease (IBD), bipolar disorder (BD), hypertension (HT), coronary artery disease (CAD), were accessed, as previously described (Wellcome Trust Case Control Consortium (WTCCC) (2007) Nature 447:661-678). For each dataset, the genotype calls generated by the Chiamo algorithm were downloaded and the default confidence score of 0.9 was applied to keep the high-quality genotype calls. In addition, the 30,956 SNP markers failing QC threshold (due to one of three criteria) were removed, as specified in the website. For each dataset, the same recommended sample exclusion criteria was observed, as specified in the various “exclusion-list” files in the data repository.

T1D GWAS Dataset from GoKinD

The Genetics of Kidneys in Diabetes (GoKinD) study (Mueller et al. (2006) J. Am. Soc. Nephrol., 17:1782-1790; Manolio et al. (2007) Nat. Genet., 39:1045-1051) T1D case data were downloaded from dbGaP (Mailman et al. (2007) Nat. Genet., 39:1181-1186). This dataset consists of T1D cases only (about half have diabetic nephropathy but half without nephropathy). Therefore, the UK Blood Service dataset from WTCCC was subsequently used as control subjects for the risk assessment sensitivity/specificity analysis. Both the case and control genotypes in this dataset were independent and not used in the prediction model building.

T1D GWAS Datasets from CHOP and Montreal

The third T1D case series used in this study was genotyped at the Children's Hospital of Philadelphia (CHOP) and a subset of this cohort has been previously described (Hakonarson et al. (2007) Nature 448:591-594). The dataset contains 1,008 T1D subjects and 1,000 control subjects. The T1D families and cases were identified through pediatric diabetes clinics at the Children's Hospital of Montreal and at CHOP. All control subjects were recruited through the Health Care Network at CHOP. The multi-dimensional scaling analysis on genotype data was used to identify subjects of genetically inferred European ancestry. All subjects were genotyped at ˜550,000 SNPs by the Illumina® HumanHap550 Genotyping BeadChip; to apply the prediction model on these subjects, genotype imputation (see below) was subsequently used to generate imputed genotypes on these subjects.

### Genotype Imputation on Illumina® Arrays

The Markov Chain Haplotyping (MACH) software (www.sph.umich.edu/csg/abecasis/MaCH/index.html) was used for genotype imputation on markers that are present in the Affymetrix array from WTCCC, but not present in the Illumina® HumanHap550K arrays used by us. The default two-step imputation procedure is adopted for imputation: (1) In the first step, 500 randomly selected subjects of European ancestry are used to estimate the best model parameters. This model includes both an estimate of the “error” rate for each marker (an omnibus parameter which captures both genotyping error, discrepancies between the imputed platform and the reference panel, and recurrent mutation) and of “crossover” rates for each interval (a parameter that describes breakpoints in haplotype stretches shared between the imputed and the reference panel). The software requires several input files for SNPs and phased haplotypes; the HapMap phased haplotypes (release 22) was used on CEU subjects, as downloaded from the HapMap database (www.hapmap.org). (2) In the second step, the optimized model parameters was used to impute the genotypes on >2 million SNP markers in HapMap data. The default Rsq threshold of 0.3 in the mlinfo file was used to flag unreliable markers used in the imputation analysis, and the posterior probability threshold of 0.9 was used to flag unreliable genotype calls. The imputed genotype data were then checked for strand orientation (since the Affymetrix genotype data from WTCCC may not align correctly with the HapMap phased genotype data) and inconsistencies were resolved using the flip function in the PLINK software (Purcell et al. (2007) Am. J. Hum. Genet., 81:559-575).

### Disease Risk Assessment Model Building

For the instant purposes, genetic profiles on p SNPs for n individuals may be summarized by an n*p matrix G=(gᵢⱼ), where gᵢⱼ denotes the genotypic value of SNP i in individual j. The genotype data are encoded by 0, 1 and 2. In genome wide association studies, data for each individual consist of a genetic profile Gᵢ=(gᵢ₁, . . . , gᵢₚ) and a disease indicator yᵢ∈{0,1}, namely, predictor variables Gᵢ and response variable yᵢ. Based on current genotyping technologies, the number of SNPs p typically can be as large as several hundred thousands, whereas the number of individuals n is several thousands in typical genetic studies. Therefore, in the comparison of prediction methods, only the list of markers reaching a pre-defined statistical threshold of association with disease was used. As a result, the number of SNPs used for disease prediction is substantially reduced to at most one or two thousands in the studies.

In machine learning, a predictor or classifier is built from past experience and is used to make predictions of unknown future. In the instant case, a trained classifier partitions the space of genetic profiles into two disjoint and exhaustive subsets, cases and controls, such that for a DNA sample with genetic profile g=(g₁, . . . , gₚ), the classifier can accurately predict if it is a case sample or a control sample. With the large amount of WTCCC case-control data available for training, a predictor with good accuracy was built that was subsequently applied on two independent test cohorts. While the challenges ahead in translating the emerging genomic knowledge into clinical practice, it is envisioned that the approach taken is an important step towards these goals.

Many classification methods have been developed and applied to various domains. No classifier could show dominant performance consistently in all applications. In the instant comparison study, the efficacy of two representative ones were compared, logistic regression (LR) and support vector machine (SVM). A logistic regression model and its variants are one of the most widely used approaches in genetic data analysis. They are simple but often provide an adequate and interpretable description of how the inputs affect the output. In addition, simpler linear methods work as well or better for microarray classification problems where the number of candidate predictors exceeds the number of samples by orders of magnitude (Dudoit et al. (2001) J. Amer. Stat. Assoc., 97:77-87). The logistic regression model was considered, which models the posterior probabilities of being a case or a control via a linear combination of gᵢ₁, . . . , gᵢₚ. Formally,

$$log \frac{Pr ( y = 1 \vert G = g )}{1 - Pr ( y = 1 \vert G = g )} = {β}_{0} + {β}_{1} {g}_{1} + … + {β}_{p} {g}_{p}$$

Under the LR model (β₀, β₁, . . . , βₚ), the probability of being cases (y=1) for a genetic profile is exp(β^{T}g)/(1+exp(β^{T}g)), where β=(β₀, β₁, . . . , βₚ) and g=(1, g₁, . . . , gₚ)^{T}. Given the training data, the LR model is fit to get a maximum likelihood estimate (MLE) of β, and this estimate can be used for future prediction. It is noted that gi∈{0,1,2} is treated as a numeric value.

The LR model has the advantage that the main effect of each SNP to the phenotypes has a linear and interpretable description. The effect of each SNP can be naturally interpreted as the increase of the log odds ratio in favor of being a case when the count of risk allele changed by 1. One caveat of using LR model in GWAS is that linkage disequilibrium dependency of input markers may make the parameter estimation unstable. To address this issue, a L\hat{0}2 regularization was imposed on the LR model building (Le Cessie et al. (1992) Appl. Stat., 41:191-201.). The LR model was implemented based on the stepPlr package in R developed by Park and Hastie (Park et al. (2008) Biostatistics 9:30-50).

The second classifier was applied is the support vector machine (SVM) algorithm (Vapnik, V. (1999) The Nature of Statistical Learning Theory: Springer; Burges, C J C (1998) Data Mining Knowl. Disc., 2:1-47). SVM is one of the most popular supervised classifiers in the field of machine learning and has been widely used in many bioinformatics applications. SVM aims to find an optimal separating hyperplane between cases and controls and this is achieved by two key factors: large margin separation and kernel functions. The classification problem is formulated by SVM as the optimization problem:

$${max C}_{β , {β}_{0} , ‖ β ‖ = 1}$$

$$subject to {{y}_{i} ( {β}^{T} g + {β}_{0} )}_{i} \geq C , i = 1 , … , N$$

When the samples are linearly separable, the optimal hyperplane is the one that creates the biggest margin C between the training points for cases and controls. When the samples in feature space (g₁, . . . , gₚ) are not linearly separable, certain overlap can be allowed by introducing the slack variable ξ=(ξ₁, ξ₂, . . , ξ_{N}) and the constraint is modified as yᵢ(βi^{T}g+β₀)ᵢ ≧C−ξᵢ ∀i, ξᵢ≧0, Σᵢ₌₁^{N} ξᵢ≦constant. More importantly, linear separability can be obtained in an expanded input feature space h(g) by using kernel functions. Specially for SVM, the explicit transformation h(g) is not needed and only knowledge of the kernel function is required K(g, g′)=<h(g), h(g′)>, which computes inner products in the expanded feature space. Two popular kernel functions in the SVM literature are:

Polynomial kernel of degree d: K(g,g′)=(κ+<g,g′>)^{d}

Radial kernel: K(g,g′)=exp(−∥g−g′∥²/ 2)

When κ=0 and d=1 , it is a special case of the polynomial kernel called the linear kernel, which operates in the original input feature space. Using non-linear kernel functions, SVM can produce nonlinear boundaries to separate two classes of objects by constructing equivalent linear boundaries in an expanded input feature space. Such transformations usually increase accuracy considerably on one hand, but on the other hand the transformations cause poor interpretability, namely, it is not clear how the inputs affect the output even though high accuracy is obtained.

In the case of disease risk assessment, SVM constructs an optimal linear boundary (prediction model) in an expanded input feature space (in this case, transformed genotype calls for a collection of SNPs). New features, or a transformation of input features (SNP genotypes), can be derived by using the kernel function (Burges, C J C (1998) Data Mining Knowl. Disc., 2:1-47), with the goal of making inputs linearly separable. However, no biological interpretation can be attached to each predictor variable (SNP) in the prediction model. The SVM model was implemented using the machine learning package e1071 in R. It is based on the popular SVM library LIBSVM (Fan et al. (2005) J. Mach. Learn. Res., 6:1889-1918). For model building, all default options were used including the radial kernel. To assess the effect of data transformation implemented in the radial kernel, the use of the linear kernel was also explored and their predictive performance was compared.

### SNP Data Processing and Coding

For the case-control datasets, to reduce the potential concern on stratification or batch effects, highly stringent quality control measures was applied to select SNPs to use in the prediction models. Several quality filters were applied, including call rate >95%, minor allele frequency>5% and Hardy-Weinberg equilibrium P-value >1×10⁻³, for the selection of SNPs. The EigenStrat algorithm (Price et al. (2006) Nat. Genet., 38:904-909) was used on genotype data, and selected subsets of SNPs reaching pre-defined P-value thresholds to build prediction models, including P<1×10⁻⁸, P<1×10⁻⁷, P<1×10⁻⁶, P<1×10⁻⁵, P<1×10⁻⁴ and P<1×10⁻³. Additionally, only autosomal markers were used in the prediction model so that the model can be applied to both genders. Finally, SNPs were removed from the training data that are not present in the testing data (for example, SNPs not in HapMap or SNPs without known dbSNP identifiers). Genotypes with missing values were imputed by sampling from the allele frequency distribution. Homozygous major allele, heterozygotes and homozygous minor allele were coded as 0, 1 and 2, respectively.

### Performance Evaluation

The simplest and most widely used method for estimating prediction error may be K-fold cross-validation. However, due to the data differential biases discussed in the paper, cross-validation approach may severely inflate the true predictive value. In K-fold cross-validation, the data is split into K roughly equal-sized parts; for the kth part of the data, the classifier is trained based on the other K-1 parts of the data and then used to predict the kth part of the data. The process is iterated for k=1, 2, . . . , K and predictions for all data are obtained. The predictions are then used for estimating prediction performance of the classifier. Typical choices of K are 5 or 10. Five-fold cross-validation was used to compare performance of the two classifiers over the seven case-control disease datasets. Specifically, accuracy, sensitivity and specificity were measured and defined as follows:
Accuracy: (TP+TN)/(TP+FN+TN+FP)
   Sensitivity: TP/(TP+FN)
   Specificity: TN/(TN+FP)
      where TP, TN, FP and FN denote the number of true positives, true negatives, false positives and false negatives, respectively. Note that since prediction algorithms typically give quantitative assessment, the default cutoff of zero point was used for the sensitivity and specificity calculation.

In addition, the performance by the area under receiver operator characteristic (ROC) curve scores (AUC scores) was also evaluated. ROC is a widely used means to evaluate the discrimination ability of binary classification methods when the test results are continuous measures. ROC curves display the relationship between sensitivity (true positive rate) and 1-specificity (false positive rate) across all possible threshold values that define the positivity of a condition (in this case, whether a subject has T1D diagnosis). The AUC scores may range from 0.5 to 1, with a higher score indicating better discriminatory power. Furthermore, to measure the performance of ;prediction model in clinical settings, the positive predictive value (PPV) and negative predictive value (NPV) were calculated, which incorporate the disease prevalence in the testing population. These two values were calculated as: PPV =sensitivity*prevalence/[sensitivity*prevalence+(1-specificity)*(1-prevalence)] and NPV=specificity*(1-prevalence)/[(1-sensitivity)*prevalence+specificity* (1-prevalence)].

### Results

### Overview of the Risk Assessment Algorithms

A machine-learning approach called Support Vector Machine (SVM, see Methods) was tested, as well as logistic regression (LR, see Methods) in order to assess individual disease risk for type 1 diabetes (T1D) using three GWAS datasets (Table 1). SVM is one of the most popular classifiers in the field of machine learning and achieves state-of-the-art accuracy in many computational biology applications (Noble, W S (2006) Nat. Biotechnol., 24:1565-1567). In essence, SVM is a supervised machine-learning algorithm that produces a linear boundary to achieve maximum separation between two classes of subjects (cases versus controls), by mathematical transformation (kernel function) of the input features (SNP genotypes) for each subject. Unlike most regression-based methods, SVM allows more input features (such as SNPs or genes) than samples, so it is particularly useful in classifying high-dimensional data, such as microarray gene expression data (Brown et al. (2000) Proc. Natl. Acad. Sci., 97:262-267). LR was also applied as a control algorithm, since it is widely used in genetic studies to model the joint effects of multiple variants. Unlike previous disease assessment studies that typically use genotype data from a handful of validated susceptibility loci, a large ensemble of SNP markers with suggestive evidence for association with T1D was examined, using a few P-value cutoff thresholds ranging from 1×10⁻³ to 1×10⁻⁸, as well as highly stringent quality control measures (see Methods). When more relaxed P-value criteria are being used, the contributing SNPs scatter across the genome; when more stringent criteria are used (P<1×10⁻⁸), only a few independent loci contribute (assuming that all MHC markers represent a single locus). Furthermore, the 45 known T1D susceptibility markers (Barrett et al. (2009) Nat. Genet., 41:703-7) were included into the prediction models to ensure that their predictive values were accounted for. Although these SNP lists may contain some false positive loci that are not genuinely associated with T1D, recent advancements in machine-learning, such as regularization, have made classifiers more tolerant to irrelevant input features (Xing et al. (2001) Feature selection for high-dimensional genomic microarray data. Proceedings of the Eighteenth International Conference on Machine Learning (ICML2001)). Since falsely associated loci from the list of predictors cannot be completely eliminated, the goal is to include them in the prediction models (using various thresholds) and then assess their influence on performance.

TABLE 1
Description of the three T1D datasets used in the study.

| | Num | Num | | |
|---|---|---|---|---|
| GWAS | of | of | Array | |
| dataset | Cases | controls | platform | Purpose |
| WTCCC- | 1,963 | 1,480 | Affymetrix | Prediction model training |
| T1D | | | Mapping | and parameter selection; |
| | | | 500K | evaluation of predictive |
| | | | | models trained on CHOP/ |
| | | | | Montreal-T1D |
| CHOP/ | 1,008 | 1,000 | Illumina ® | Evaluation of predictive |
| Montreal- | | | HumanHap | models trained on WTCCC- |
| T1D | | | 550 | T1D, using whole-genome |
| | | | | imputed genotype data |
| GoKinD- | 1,529 | 1,458 | Affymetrix | Evaluation of predictive |
| T1D | | | Mapping | models trained on WTCCC- |
| | | | 500K | T1D or CHOP/Montreal-T1D |

Evaluation of Risk Assessment Models by Within-Study Cross-Validation

To evaluate the sensitivity of various risk assessment models on the number of predictor variables and the parameters of the models, five-fold cross-validation experiments was performed on the WTCCC-T1D dataset. During each cross-validation, 80% of the samples (both cases and controls) were used to select a subset of SNPs as predictors (see Methods), train a prediction model, and then test on the remaining 20% of the samples. The results from a within-study cross-validation do not completely reflect the true performance of risk assessment, but can help select relevant parameters or thresholds to use. The AUC (area under ROC curve) score was used to evaluate the performance of risk assessment: the value ranges from 0.5 to 1, with a higher number indicating better discriminative power between cases and controls. Under various thresholds for SNP selection, the SVM algorithm consistently out-performed LR, achieving the highest AUC score of ˜0.9 (Table 2). The best performance seems to be achieved when a P-value cutoff of 1×10⁻⁵ is used for selecting SNPs for SVM model training, corresponding to 399-443 SNPs in five cross-validation experiments.

TABLE 2
Evaluation of risk assessment models on the WTCCC-
T1D dataset by five-fold cross-validation.

| | AUC¹ | Sensitivity³ | Specificity³ | Min | Max |
|---|---|---|---|---|---|
| SNP selection | (SD²) | (SD²) | (SD²) | #SNP | #SNP |
| | SVM (support vector machine) | | | | |
| P < 1 × 10⁻⁸ | 0.89 (0.017) | 0.87 (0.018) | 0.75 (0.041) | | |
| P < 1 × 10⁻⁷ | 0.89 (0.018) | 0.87 (0.024) | 0.75 (0.036) | | |
| P < 1 × 10⁻⁶ | 0.89 (0.018) | 0.88 (0.019) | 0.74 (0.041) | | |
| P < 1 × 10⁻⁵ | 0.89 (0.013) | 0.88 (0.013) | 0.73 (0.041) | | |
| P < 1 × 10⁻⁴ | 0.88 (0.012) | 0.87 (0.021) | 0.73 (0.026) | | |
| P < 1 × 10⁻³ | 0.86 (0.010) | 0.85 (0.020) | 0.69 (0.015) | | |
| | LR (logistic regression) | | | | |
| P < 1 × 10⁻⁸ | 0.89 (0.016) | 0.86 (0.026) | 0.75 (0.035) | 240 | 280 |
| P < 1 × 10⁻⁷ | 0.88 (0.018) | 0.86 (0.034) | 0.76 (0.031) | 286 | 328 |
| P < 1 × 10⁻⁶ | 0.89 (0.022) | 0.86 (0.033) | 0.76 (0.044) | 328 | 372 |
| P < 1 × 10⁻⁵ | 0.88 (0.014) | 0.85 (0.028) | 0.75 (0.037) | 399 | 433 |
| P < 1 × 10⁻⁴ | 0.87 (0.011) | 0.84 (0.016) | 0.75 (0.030) | 519 | 558 |
| P < 1 × 10⁻³ | 0.80 (0.009) | 0.77 (0.040) | 0.69 (0.025) | 1007 | 1085 |

¹area under receiver operating characteristic curve.
²standard deviation.
³sensitivity and specificity were calculated with default cutoff of zero point.

### Evaluation of Risk Assessment Models on Independent Datasets

To assess the prediction model in an unbiased way, it is important that independent datasets from different sources be evaluated. This is a practical concern for all GWAS, since the SNPs detected from the training dataset may be spuriously associated with the disease, when cases and controls undergo different DNA preparation protocols (Plagnol et al. (2007) PLoS Genet 3:e74), when cases and controls are genotyped in different batches, when population stratification is present (Cardon (2003) Lancet 361:598-604) or when cases share other traits that are unrelated to the disease of interest (for example, cases often have a higher average body mass index than controls when studying T2D (Frayling et al. (2007) Science 316:889-894). A within-study cross-validation design is not able to adjust for these potential biases which are present in both the training and testing data; therefore, the risk assessment models parameterized from the WTCCC-T1D dataset were tested on additional GWAS datasets (Table 1).

Since the CHOP/Montreal-T1D dataset was genotyped using the Illumina® platform, whole-genome imputed genotypes were generated using MACH and then shared markers present on the Affymetrix array were utilized for the risk assessment. Additionally, a third GWAS dataset from the Genetics of Kidneys in Diabetes consortium (GoKinD) was used, which was genotyped on the same platform as the WTCCC-T1D data. Shared markers for the risk assessment were used. Since this dataset does not contain control subjects, this dataset was supplemented with control subjects from the UK Blood Service (UKBS) collection (a subset of samples from WTCCC not used in the training phase). Similar to the analysis presented above, the P-value cutoff thresholds were varied and the results were summarized for each threshold.

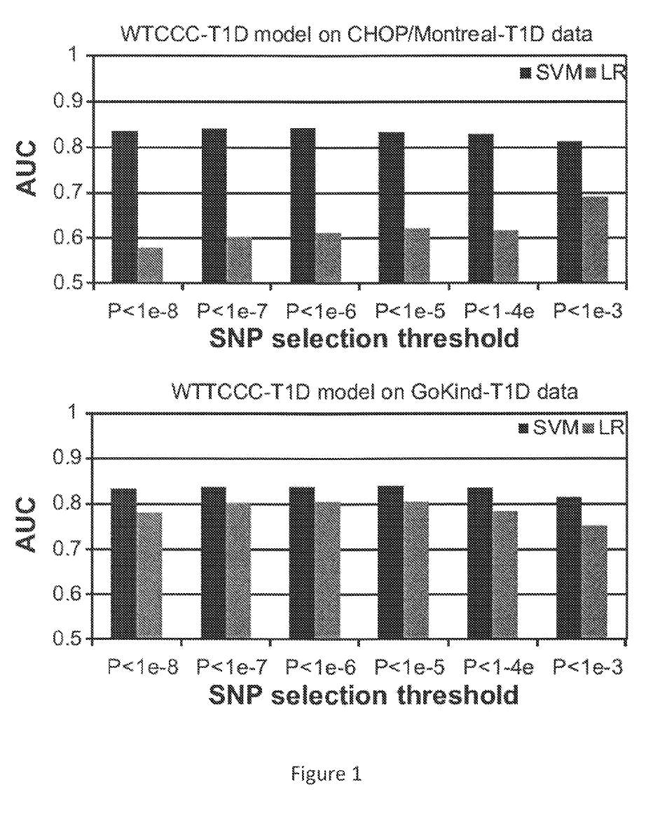

It was found that the AUC score is 0.83 and 0.84 for the CHOP/Montreal-T1D and GoKinDT1D datasets, respectively, when SNPs with P-value cutoff of 1×10⁻⁵ were used in the SVM model (FIG. 1, Table 3 and Table 4). These values are notably lower than those obtained in cross-validation experiments, suggesting that data differential biases might lead to the inflated performance measures for both SVM and LR seen in Table 2. Nevertheless, SVM consistently achieves higher accuracy than LR, and the AUC scores in both datasets still indicate reasonably good performance. Unlike the previous cross-validation results in Table 2, it was found that SVM demonstrate more clear advantage over LR when evaluated by between-study validation. This suggests that SVM may be less susceptible to differential biases than LR through improved utilization of a subset of SNPs, so the differences in performance is less when comparing results generated on independent datasets versus those generated by cross-validation. Notably, the performance advantage of SVM over LR is less obvious, when models were tested on the GoKind-T1D dataset. This could be due to several reasons: First, the control group for the GoKind-T1D dataset was generated at the same site as the WTCCC-T1D dataset, which may introduce differential biases that are shared between the two datasets, with LR being more susceptible to biases than SVM. Second, the CHOP/Montreal-T1D dataset was imputed for proper genotype matching, which may lead to systematic differences from the WTCCC-T1D data from some less well imputed markers due to platform differences. Third, the GoKind-T1D dataset contains markers passing QC in both the WTCCC study and the GoKind study, so they represent a subset of higher-quality markers, making experiments on GoKind-T1D less susceptible to biases. To further investigate this, the experiments of training models were re-performed on WTCCC-T1D and tested on CHOP/Montreal-T1D, using makers that passed QC in GoKind-T1D data (P<1×10⁻⁵ threshold, 409 markers, as opposed to 478 markers): the AUC score for LR increased to 0.82 but remained at 0.84 for SVM, suggesting that inclusion of lower-quality markers led to degraded performance for LR while the impact was less for SVM.

TABLE 3
Prediction performance of the WTCCC-T1D trained model on the
CHOP/Montreal-T1D datasets. These values were used in FIG. 1.

| Algorithm | P Cutoff | 1 × 10⁻⁸ | 1 × 10⁻⁷ | 1 × 10⁻⁶ | 1 × 10⁻⁵ | 1 × 10⁻⁴ | 1 × 10⁻³ |
|---|---|---|---|---|---|---|---|
| | #SNPs | 312 | 365 | 417 | 478 | 632 | 1145 |
| SVM | AUC | 0.835 | 0.84 | 0.841 | 0.833 | 0.828 | 0.812 |
| | Sensitivity | 0.776 | 0.77 | 0.761 | 0.772 | 0.767 | 0.765 |
| | specificity | 0.758 | 0.762 | 0.758 | 0.74 | 0.75 | 0.702 |
| LR | AUC | 0.58 | 0.605 | 0.613 | 0.622 | 0.617 | 0.693 |
| | Sensitivity | 0.517 | 0.536 | 0.549 | 0.546 | 0.551 | 0.599 |
| | specificity | 0.588 | 0.604 | 0.601 | 0.62 | 0.588 | 0.686 |

TABLE 4
Prediction performance of the WTCCC-T1D trained model on the
GoKind-T1D datasets. These values were used in FIG. 1.

| Algorithm | P Cutoff | 1 × 10⁻⁸ | 1 × 10⁻⁷ | 1 × 10⁻⁶ | 1 × 10⁻⁵ | 1 × 10⁻⁴ | 1 × 10⁻³ |
|---|---|---|---|---|---|---|---|
| | #SNPs | 267 | 313 | 358 | 409 | 539 | 1004 |
| SVM | AUC | 0.832 | 0.838 | 0.837 | 0.839 | 0.834 | 0.814 |
| | Sensitivity | 0.779 | 0.792 | 0.793 | 0.791 | 0.788 | 0.795 |
| | specificity | 0.716 | 0.719 | 0.716 | 0.717 | 0.708 | 0.669 |
| LR | AUC | 0.781 | 0.803 | 0.805 | 0.805 | 0.786 | 0.753 |
| | Sensitivity | 0.708 | 0.734 | 0.732 | 0.723 | 0.716 | 0.707 |
| | specificity | 0.679 | 0.695 | 0.706 | 0.709 | 0.713 | 0.663 |

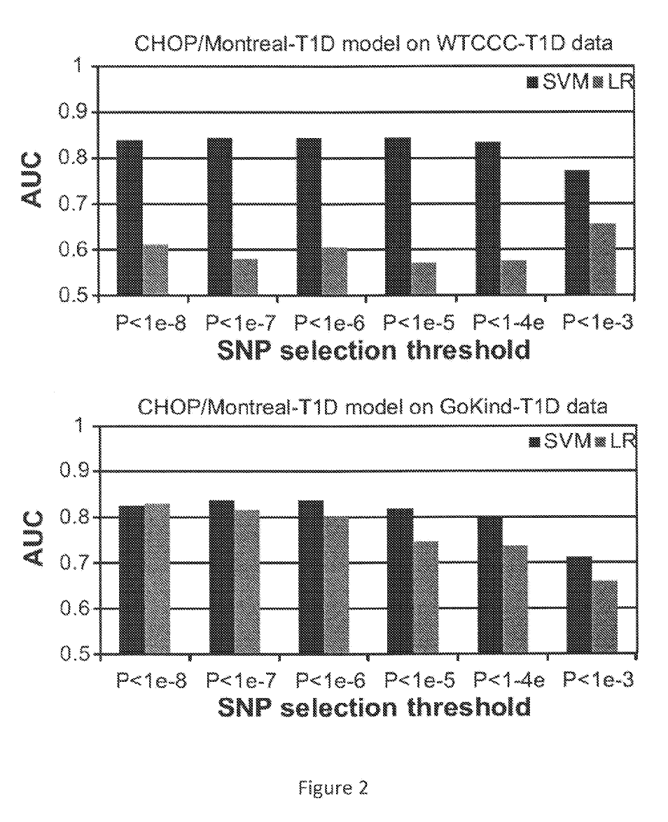

Furthermore, it was investigated how the algorithms performed when the models were trained on an independent dataset with different size and ascertainment schema. As such, prediction models were built from the imputed CHOP/Montreal-T1D dataset using markers that are present on the Affymetrix arrays, and then the model performance was evaluated on the WTCCCT1D and the GoKind-T1D data (FIG. 2, Table 5 and Table 6). Despite the use of a different training dataset, SVM still demonstrated an advantage over LR, with an AUC score of 0.85 on WTCCC-T1D and 0.84 on GoKind-T1D datasets, respectively, when a P<1×10⁻⁶ threshold is used for SNP selection. Altogether, these results indicate that an SVM-based risk assessment algorithm can accommodate differences in training data and can generate consistent, robust results across different datasets.

TABLE 5
Prediction performance of the CHOP/Montreal-T1D trained model
on the WTCCC-T1D datasets. These values were used in FIG. 2.

| Algorithm | P Cutoff | 1 × 10⁻⁸ | 1 × 10⁻⁷ | 1 × 10⁻⁶ | 1 × 10⁻⁵ | 1 × 10⁻⁴ | 1 × 10⁻³ |
|---|---|---|---|---|---|---|---|
| | #SNPs | 192 | 235 | 280 | 340 | 445 | 1098 |
| SVM | AUC | 0.839 | 0.844 | 0.845 | 0.843 | 0.834 | 0.771 |
| | Sensitivity | 0.876 | 0.876 | 0.864 | 0.855 | 0.&33 | 0.724 |
| | specificity | 0.632 | 0.646 | 0.653 | 0.666 | 0.667 | 0.676 |
| LR | AUC | 0.614 | 0.582 | 0.607 | 0.573 | 0.578 | 0.655 |
| | Sensitivity | 0.616 | 0.591 | 0.611 | 0.552 | 0.554 | 0.581 |
| | specificity | 0.535 | 0.507 | 0.536 | 0.532 | 0.551 | 0.629 |

TABLE 6
Prediction performance of the CHOP/Montreal-T1D trained model
on the GoKind-T1D datasets. These values were used in FIG. 2.

| Algorithm | P Cutoff | 1 × 10⁻⁸ | 1 × 10⁻⁷ | 1 × 10⁻⁶ | 1 × 10⁻⁵ | 1 × 10⁻⁴ | 1 × 10⁻³ |
|---|---|---|---|---|---|---|---|
| | #SNPs | 166 | 201 | 237 | 295 | 392 | 998 |
| SVM | AUC | 0.826 | 0.837 | 0.838 | 0.819 | 0.801 | 0.713 |
| | Sensitivity | 0.83 | 0.825 | 0.815 | 0.78 | 0.758 | 0.611 |
| | specificity | 0.688 | 0.697 | 0.709 | 0.711 | 0.711 | 0.687 |
| LR | AUC | 0.83 | 0.816 | 0.802 | 0.747 | 0.737 | 0.66 |
| | Sensitivity | 0.788 | 0.806 | 0.754 | 0.695 | 0.689 | 0.532 |
| | specificity | 0.709 | 0.682 | 0.689 | 0.671 | 0.66 | 0.666 |

Predictive Models have High Specificity for T1D

The analyses of three GWAS datasets demonstrate that the SVM-based prediction model is highly reliable in separating T1D cases from control subjects, but a remaining concern is whether the model is specific to T1D, that is, does it tend to predict patients with other diseases as potential T1D cases? To address this concern, the same risk assessment model trained on WTCCC-T1D dataset was applied on six other disease cohorts from WTCCC, including bipolar disorder (BD), coronary heart disease (CAD), Crohn's disease (CD), hypertension (HT), rheumatoid arthritis (RA) and type 2 diabetes (T2D). This analysis is especially interesting, since these diseases include a different subtype of diabetes and two autoimmune diseases (CD and RA), which may share some susceptibility loci with T1D.

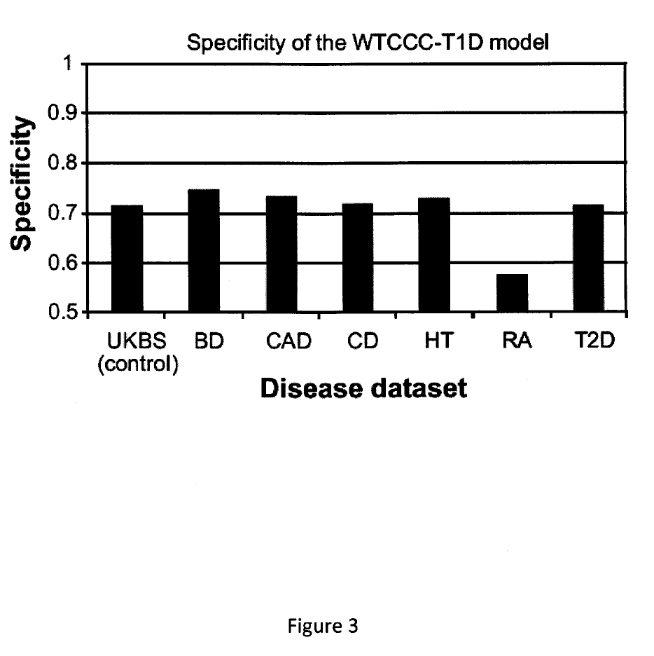

By testing the SVM-based prediction model for T1D on other six disease cohorts, it was found that with the exception of RA, the specificity values are good, ranging from 71.6% for T2D to 74.8% for BD (FIG. 3). The specificity for RA, an autoimmune disease with a large genetic susceptibility component from the MHC region, is 57.6%, confirming that T1D and RA do share some genetic risk factors and susceptibility pathways. Besides MHC, the PTPN22 locus on 1p13 is well known to contribute to both T1D and RA (Begovich et al. (2004) Am. J. Hum. Genet., 75:330-337; Bottini et al. (2004) Nat. Genet., 36:337-338), and the WTCCC study reported three additional shared susceptibility loci (IL2RA on 10p15, PTPN2 on 18p11 and chromosome 12q14 region) (Wellcome Trust Case Control Consortium (2007) Nature 447:661-678). For other diseases, these specificity values are at similar range or slightly higher than that for the UK Blood Service (UKBS) control cohort, so a patient affected by diseases unrelated to T1D is not more likely to be predicted as a T1D patient, compared to a control subject. In conclusion, the analyses suggest that the risk assessment model built for T1D is specific to that disease.

### Understanding the Behavior of the Risk Assessment Models

To investigate in depth why the SVM algorithm works in the setting of T1D risk assessment, several different forms of the risk assessment models were evaluated by modifying the predictors or the model parameters. The following six different types of analyses help better understand the source of the improved performance of the SVM algorithm.

1) It was found that elimination of SNPs in the MHC region severely deteriorate the performance of disease risk assessment. Since a large fraction of the genetic contribution to T1D can be explained by risk alleles in the major histocompatibility complex (MHC) region (-50%) (Risch, N. (1987) Am. J. Hum. Genet., 40:1-14), it was tested whether using SNPs outside of the MHC region had any predictive value. This analysis helped identify the relative contribution of MHC-linked SNPs with major effects and other SNPs with moderate effects on disease risks. For this analysis, all of the SNPs located from 25 Mb to 34 Mb on chromosome 6 were removed. This genomic span is larger than the actual MHC region in order to ensure that SNPs outside MHC but in linkage disequilibrium (LD) with MHC markers are not used in the prediction model. With the P<1×10⁻⁵ threshold, a large proportion of the SNPs (˜83%) were removed from the prediction models. The performance of the SVM and LR algorithms was then tested using the same approach described above. As expected, using SNPs outside of the MHC region do not confer satisfactory performance in disease risk assessment. For example, for the GoKindT1D data, using SNPs with P<1×10⁻⁵, the AUC score for SVM dropped from 0.84 to 0.64, whereas the AUC score for LR dropped from 0.81 to 0.65. Similar results were obtained for the CHOP/Montreal-T1D dataset. Therefore, elimination of SNPs with major effects severely attenuates the performance of disease risk assessment; on the other hand, the analyses also confirmed that SNPs outside the MHC region do explain a portion of the genetic susceptibility to T1D and can provide complementary information for risk assessment. These results also suggest that whole-genome genotypes provide more information than the costly HLA-typing techniques used in the clinical settings, even for the purpose of risk assessment of MHC-linked diseases such as T1D.

2) It was found that pruned sets of independent markers lead to worse performance. The risk assessment model used sets of markers reaching pre-defined thresholds, which may include correlated markers. The SVM algorithm is inherently capable of handling the inter-marker correlation structure, whereas regularization techniques (Le Cessie et al. (1992) Appl. Stats., 41:191-201) were used in the LR model for addressing this problem. Stepwise regression model was not used because it is highly unstable when the number of predictor variables is large. Since many markers are in high LD with each other, this list can be pruned to generate a smaller set of markers that have pairwise r² less than a certain threshold. Intuitively, using fewer markers should lead to information loss and therefore lower predictive power, but it was desirable to specifically quantify this magnitude of loss. SVM-based prediction models were trained on the WTCCC-T1D dataset using SNPs with P<1×10⁻⁵ that were pruned by various thresholds: when r² threshold of 0.1, 0.2, 0.5 and 0.8 were used, the AUC scores in the testing dataset were 0.65 (63 SNPs), 0.76 (75 SNPs), 0.79 (153 SNPs) and 0.83 (268 SNPs), respectively. The P<1×10⁻⁻⁸ criteria was then used to select SNP markers, and then the same set of computational experiment was performed again: when r² threshold of 0.1, 0.2, 0.5 and 0.8 were used, the AUC scores in the testing dataset were 0.67 (55 SNPs), 0.76 (60 SNPs), 0.79 (113 SNPs) and 0.82 (184 SNPs), respectively. Altogether, the analyses indicates that the use of independent markers does indeed lose information which is important for risk assessment. Therefore, many previous studies that use only the single most significant SNP per associated loci did not capitalize on all the available genotype information in an optimal manner.

3) It was found that radial kernel performs better than linear kernel in SVM. The SVM algorithm that was used adopted a default radial kernel to transform genotype scores (see Methods), as it is a widely used kernel in most SVM applications. To determine if the data transformation leads to better performance, the SVM algorithm was also evaluated without any transformation, that is, with a linear kernel. Similar to previous experiments, SVM-based assessment models were trained on the WTCCC-T1D dataset using SNPs with P<1×10⁻⁵. It was found that the AUC scores of SVM using linear model are less than those with radial kernel for the GoKind-T1D dataset (0.77 vs 0.84), indicating that linear combination of predictors (SNPs) is less optimal than higher-order transformation of predictors when separating cases versus controls using SNP genotypes. Similar results were obtained for the CHOP/Montreal-T1D dataset. These observations are consistent with recent findings on the genetic interactions between MHC loci and non-MHC loci for conferring T1D risk (Barrett et al. (2009) Nat. Genet., 41:703-7). However, unlike LR, the SVM model suffers from poor interpretability, that is, one cannot identify specific pair of SNPs that interact with each other from the model parameters. Additionally, it is notable that two types of interactions may be important contributors to the risk assessment: statistical interactions between unlinked SNPs (that contribute to liability scale in a non-additive fashion), as well as haplotype interactions (correlated SNPs that are on the same haplotype and are not captured by a model with additive predictors).

4) The relative effect of modeling correlated SNPs was investigated in MHC and non-MHC regions. The results demonstrate that handling of interactions between SNPs, as well as utilizing SNPs with major effects in the MHC region, are important for risk assessment, but their relative contribution is unknown. To test the effect of incorporating non-MHC loci and modeling correlated SNPs for the performance of LR and SVM, respectively, several variations of the pruned analysis were performed, with the P<1×10⁻⁵ threshold for selecting SNPs and with pairwise r²<0.2 threshold for pruning independent sets of SNPs in GoKind-T1D dataset (Table 7). First, a pruned list of MHC SNPs only was used, so only independent markers contribute to risk assessment: the AUC for LR and SVM is 0.70 and 0.74, respectively. The decreased performance could be due to the inability to model interaction effects between correlated SNPs, but it also could be due to the (unknown) causal variants being tagged less well in the pruned set. Second, a pruned list of MHC SNPs plus all non-MHC SNPs was used: the AUC for LR and SVM is 0.74 and 0.75, respectively, indicating that additional non-MHC loci contribute to improved performance but the effects are more obvious for LR. Third, MHC SNPs only but without pruning was used: the AUC for LR and SVM is 0.78 and 0.81, respectively, indicating that both LR and SVM benefit from incorporating correlated SNPs within MHC, which play a more prominent role in the risk modeling than non-MHC markers. Altogether, these analyses indicate that a key contributor to the performance of the SVM algorithm is the better modeling of LD structure among MHC SNPs.

TABLE 7
Comparative analysis of prediction models
by including different sets of markers.

| | # | AUC¹ | AUC¹ |
|---|---|---|---|
| Marker selection (P < 1 × 10⁻⁵) | markers | (LR) | (SVM) |
| All (MHC and non-MHC) SNPs | 409 | 0.81 | 0.84 |
| MHC SNPs | 338 | 0.78 | 0.81 |
| Non-MHC SNPs | 71 | 0.65 | 0.64 |
| Pruned MHC and non-MHC | 82 | 0.74 | 0.76 |
| SNPs² | | | |
| Pruned MHC SNPs² | 27 | 0.70 | 0.74 |
| Pruned MHC SNPs² and not | 98 | 0.74 | 0.75 |
| pruned non-MHC SNPs | | | |

¹area under receiver operating characteristic curve.
²SNPs are pruned using pairwise r² threshold of 0.2.

5) It was found that an alternative allele coding scheme without assuming genetic model has similar results. In the previous analysis, for each SNP, the three different genotypes (homozygous major allele, heterozygotes, homozygous minor allele) were coded as 0, 1 and 2, respectively. To investigate the sensitivity of prediction models on allele coding, an alternative coding scheme was explored, by generating two dummy variables (0 or 1) for each SNP, indicating the presence or absence of an allele. This coding scheme effectively doubles the number of predictor variables, but without assuming an additive risk model for each SNP. The new coding scheme was tested on the GoKind-T1D dataset, and it was found that the AUC score remained the same at 0.84. For the CHOP/Montreal-T1D dataset, the AUC Score slightly decreased from 0.83 to 0.82. Therefore, relaxing genetic model assumptions do not appear to have a major impact on the performance of risk models.

6) It was found that the collection of known T1D susceptibility loci has poor performance. Recent progress with GWAS has enabled the identification of dozens of confirmed and replicated T1D susceptibility loci (Cooper et al. (2008) Nat. Genet., 40:1399-1401; Barrett et al. (2009) Nat Genet., 41:703-7). As a negative control experiment, the performance of risk assessment was tested using only established susceptibility loci. This analysis is similar to the several previously published T2D risk assessment studies, in that the prediction model only considers known information, while ignoring other potentially associated loci. Risk assessment models were built around the WTCCC-T1D dataset, using 45 known T1D susceptibility SNPs compiled from a recent meta-analysis (Barrett et al. (2009) Nat Genet., 41:703-7), after excluding one locus on chromosome X (Table 8). Note that only one representative SNP from the MHC region is used in the assessment models. For the SVM algorithm, the AUC scores are 0.66 for the GoKind-T1D dataset and 0.65 for the CHOP/Montreal-T1D dataset, indicating a limited value of risk assessment using a reduced number of validated SNPs. For the LR algorithm, the AUC scores are 0.68 for both the GoKind-T1D and the CHOP/Montreal-T1D datasets, which are slightly higher than those obtained using the SVM algorithm. Nevertheless, the relatively modest performance is not unexpected, and echoes what has already been observed in T2D disease assessment studies. Collectively, this analysis confirms that one of the keys to success is the use of a large ensemble of loci associated to the disease of interest, at the cost of including potential false positive loci.

TABLE 7
A list of 46 previously validated T1D susceptibility loci reported in the
meta-analysis by Barrett et al. The chromosome X marker is not used in
the study. The INS locus is not well covered by the Affymetrix array.

| Barrett et al SNP | WTCCC SNP | r2 | Chr | LD region | Gene or Num of genes |
|---|---|---|---|---|---|
| rs2269241 | rs2269241 | 1 | 1p31.3 | 63.87-63.94 | PGM1 |
| rs2476601 | rs6679677 | 1 | 1p13.2 | 113.62-114.46 | PTPN22 |
| rs2816316 | rs1323296 | 1 | 1q31.2 | 190.73-190.82 | RGS1 |
| rs3024505 | rs3024505 | 1 | 1q32.1 | 204.87-205.12 | IL10 |
| rs1534422 | rs10175906 | 0.72 | 2p25.1 | 12.53-12.60 | (0) |
| rs917997 | rs2041756 | 1 | 2q12.1 | 102.22-102.58 | IL18RAP |
| rs1990760 | rs7608315 | 0.59 | 2q24.2 | 162.67-163.10 | IFIH1 |
| rs3087243 | rs3087243 | 1 | 2q33.2 | 204.38-204.53 | CTLA4 |
| rs11711054 | rs6441961 | 1 | 3p21.31 | 45.96-46.63 | CCR5 |
| rs10517086 | rs17698094 | 0.54 | 4p15.2 | 25.64-25.75 | (0) |
| rs4505848 | rs17388568 | 0.83 | 4q27 | 123.13-123.83 | IL2 |
| rs6897932 | rs6897932 | 1 | 5p13.2 | 35.84-36.07 | IL7R |
| rs9268645 | rs9268645 | 1 | 6p21.32 | 24.70-34.00 | MHC |
| rs11755527 | rs11755527 | 1 | 6q15 | 90.86-91.10 | BACH2 |
| rs9388489 | rs9388489 | 1 | 6q22.32 | 126.48-127.46 | C6orf173 |
| rs2327832 | rs2327832 | 1 | 6q23.3 | 137.80-138.40 | TNFAIP3 |
| rs1738074 | rs2016588 | 0.57 | 6q25.3 | 159.13-159.62 | TAGAP |
| rs7804356 | rs10486478 | 1 | 7p15.2 | 26.62-27.17 | (10) |
| rs4948088 | rs10486748 | 0.41 | 7p12.1 | 50.87-51.64 | COBL |
| rs7020673 | rs7020673 | 1 | 9p24.2 | 4.22-4.31 | GLIS3 |
| rs12251307 | rs12251307 | 1 | 10p15.1 | 6.07-6.24 | IL2RA |
| rs11258747 | rs11258747 | 1 | 10p15.1 | 6.48-6.59 | PRKCQ |
| rs10509540 | rs11816865 | 0.66 | 10q23.31 | 90.00-90.27 | C10orf59 |
| rs7111341 | rs6578252 | 0.07 | 11p15.5 | 2.02-2.26 | INS |
| rs4763879 | rs4763879 | 1 | 12p13.31 | 9.51-9.87 | CD69 |
| rs2292239 | rs2292239 | 1 | 12q13.2 | 54.64-55.09 | ERBB3 |
| rs1678536 | rs1678542 | 0.62 | 12q13.3 | 55.27-56.82 | Multiple |
| rs3184504 | rs1265566 | 0.25 | 12q24.12 | 109.77-111.72 | SH2B3 |
| rs1465788 | rs1465788 | 1 | 14q24.1 | 68.24-68.39 | (2) |
| rs4900384 | rs4900384 | 1 | 14q32.2 | 97.43-97.60 | (0) |
| rs3825932 | rs3825932 | 1 | 15q25.1 | 76.77-77.05 | CTSH |
| rs12708716 | rs12708716 | 1 | 16p13.13 | 10.92-11.56 | CLEC16A |
| rs12444268 | rs12444268 | 1 | 16p12.3 | 20.17-20.28 | (2) |
| rs4788084 | rs151181 | 0.9 | 16p11.2 | 28.19-28.94 | IL27 |
| rs7202877 | rs4536500 | 0.64 | 16q23.1 | 73.76-74.09 | (7) |
| rs16956936 | rs16956936 | 1 | 17p13.1 | 7.56-7.66 | (2) |
| rs2290400 | rs1008723 | 1 | 17q12 | 34.63-35.51 | ORMDL3 |
| rs7221109 | rs7221109 | 1 | 17q21.2 | 35.95-36.13 | (3) |
| rs1893217 | rs16939895 | 0.69 | 18p11.21 | 12.73-12.92 | PTPN2 |
| rs763361 | rs1788101 | 0.18 | 18q22.2 | 65.63-65.72 | CD226 |
| rs425105 | rs4804000 | 0.69 | 19q13.32 | 51.84-52.02 | (5) |
| rs2281808 | rs2281808 | 1 | 20p13 | 1.44-1.71 | (3) |
| rs11203203 | rs11203203 | 1 | 21q22.3 | 42.68-42.76 | UBASH3A |
| rs229541 | rs229541 | 1 | 22q13.1 | 35.90-36.00 | C1QTNF6 |
| rs5753037 | rs41176 | 1 | 22q12.2 | 28.14-29.00 | (14) |
| rs2664170 | | | Xq28 | 153.48-154.10 | (16) |

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of determining a set of predictive markers for a disease or disorder, said method comprising:
A) obtaining a genome wide association studies dataset;
B) selecting those markers within the dataset that have a P-value of less than 1×10⁻⁴; and
C) applying a support vector machine algorithm to the selected markers, thereby obtaining a set of predictive markers for said disease or disorder.

2. The method of claim 1, wherein the P-value is less than 1×10⁻⁵.

3. The method of claim 1, wherein the P-value is less than 1×10⁻⁶.

4. The method of claim 1, wherein said disease or disorder is selected from the group consisting of type 1 diabetes, schizophrenia, autism, inflammatory bowel disease, Crohn's Disease, colitis, inflammatory/autoimmune diseases, rheumatoid arthritis, lupus, celiac disease, and asthma.

5. The method of claim 4, wherein said disease or disorder is type 1 diabetes.

6. A method of diagnosing a disease or disorder in a patient, said method comprising:
A) obtaining a biological sample from said patient;
B) determining the presence or absence of a set of predictive markers for the disease or disorder in said biological sample; and
C) applying a support vector machine algorithm to the results obtained in step B), thereby predicting the risk said patient has in developing said disease or disorder.

7. The method of claim 6, wherein said disease or disorder is type 1 diabetes.

8. The method of claim 6, wherein step B) is performed by hybridizing the nucleic acids of said biological sample with said set of predictive markers.

9. The method of claim 8, wherein said set of predictive markers are on a microarray.

10. The method of claim 6, wherein said disease or disorder is selected from the group consisting of type 1 diabetes, autism, schizophrenia, rheumatoid arthritis, inflammatory bowel disease, Crohn's disease, inflammatory/autoimmune diseases, celiac disease, colitis, lupus, and asthma.

11. A microarray comprising the set of predictive markers obtained by the method of claim 1.

12. A microarray comprising a set of predictive markers for type 1 diabetes, wherein said microarray comprises oligonucleotide probes which specifically hybridize to or comprise at least 80% of the 478 SNPs presented in FIG. 5.

13. The microarray of claim 12, wherein said microarray comprises oligonucleotide probes which are completely complementary to the 478 SNPs presented in FIG. 5.

14. The microarray of claim 12, wherein said microarray comprises oligonucleotide probes which specifically hybridize to or comprise at least 90% of the 478 SNPs provided in FIG. 5.

15. The microarray of claim 12, wherein said microarray comprises oligonucleotide probes which comprise the 478 SNPs presented in FIG. 5.

16. A kit comprising the microarray of claim 11.

17. A kit comprising the microarray of claim 12.
